(19) United States
(12) Patent Application Publication
(10) Pub. No.: US 2009/0054799 A1
(43) Pub. Date: Feb. 26, 2009

(54) Biosensor system with a multifunctional portable electronic device

(76) Inventors: Joan K. Vrtis, Mesa, AZ (US); Barbara E. Landini, Mesa, AZ (US)

Correspondence Address: GREENBERG TRAURIG (PHX), INTELLECTUAL PROPERTY DEPARTMENT, 2450 COLORADO AVENUE , SUITE 400E, SANTA MONICA, CA, 90404, US

(21) Appl. No.: 12/228,046

(22) Filed: Aug. 8, 2008

Related U.S. Application Data
(60) Provisional application No. 60/963,950, filed on Aug. 8, 2007.

(51) Int. Cl. A61B 5/08

(52) U.S. Cl. 600/532

(57) ABSTRACT

Some embodiments of the invention provide a biosensor system with a multifunctional portable electronic device for use by an individual. The biosensor system includes a breath delivery system with a breath sensor capable of detecting an analyte in the individual's breath. The system also includes a portable electronic device capable of receiving breath analyte data from the breath sensor and blood glucose data or other types of personal health data. The portable electronic device is capable of storing, analyzing, and/or transmitting the breath analyte data and the blood glucose data or the other types of personal health data.

### RELATED APPLICATIONS

This application claims priority under 35 U.S.C. § 119 to U.S. Provisional Patent Application No. 60/963,950 filed on Aug. 8, 2007, the entire contents of which is incorporated herein by reference.

### BACKGROUND

The current state of the art for breath sensing, measuring, and analyzing of breath analytes is a gas chromatograph or other larger, lab-scale devices. Other current state of the art is the incorporation of blood glucose monitoring into a cellular phone, personal digital assistant, or other portable electronic device where data signal transmission occurs. In this current art, blood (a liquid biomaterial) is required to initiate the sensor. In this current art, the device can sense and measure only a single biomaterial.

### SUMMARY

In light of the limitations discuss above, embodiments of the invention expand the single functionality device to detect at least two biomarkers with at least one being a breath biomarker. Some embodiments of the invention provide a biosensor system for use by an individual. The system includes a breath delivery system with a breath sensor capable of detecting an analyte in the individual's breath. The system also includes a portable electronic device capable of receiving breath analyte data from the breath sensor and blood glucose data or other types of personal health data. The portable electronic device is capable of storing, analyzing, and/or transmitting the breath analyte data and the blood glucose data or other types of personal health data.

Other aspects of the invention will become apparent by consideration of the detailed description and accompanying drawings.

### BRIEF DESCRIPTION OF THE DRAWINGS

FIG. 1 is a schematic illustration of a biosensor system according to one embodiment of the invention.

FIGS. 2A-2D are perspective views of a biosensor system according to one embodiment of the invention.

FIG. 3 is a schematic illustration of a breath delivery system being inserted into a portable electronic device according to one embodiment of the invention.

FIG. 4 is a schematic illustration of a breath sensor electrically embedded into a portable electronic device according to one embodiment of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings, whether electrical or mechanical. Further, “connected” and “coupled” are not restricted to physical or mechanical connections or couplings.

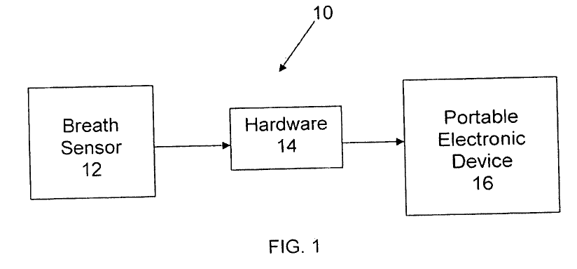

FIG. 1 illustrates a biosensor system 10 according to one embodiment of the invention. The biosensor system 10 can include a breath sensor 12, any necessary hardware 14, and a portable electronic device 16. Some embodiments of the invention provide a breath sensor 12 in the form of an electrochemical biosensor that detects one or more analytes in an individual's breath specific to the human body's metabolic state or disease state. An example of a metabolic state is fat catabolism and examples of disease states are hyperthyroidism, ketoacidosis, and diabetes. In these examples, the breath analyte is acetone. The breath sensor 12 can generate breath analyte data that can be received by the portable electronic device 16. The breath sensor 12 can be mechanically and electrically embedded, or mechanically or electrically integrated, into the portable electronic device 16.

The portable electronic device 16 can be capable of receiving the breath analyte data from the breath sensor 12. The portable electronic device 16 can also be capable of storing, analyzing, and/or transmitting the breath analyte data. The portable electronic device 16 can be a laptop, a cellular phone, a personal digital assistant, a pocket personal computer, an iPhone®, an iPod®, etc. In some embodiments, the portable electronic device 16 can transmit and receive the breath analyte data. In addition, the portable electronic device 16 can be an apparatus designed specifically for medical uses, such as that disclosed in U.S. Pat. No. 7,364,551 issued on Apr. 29, 2008, the entire contents of which is herein incorporated by reference.

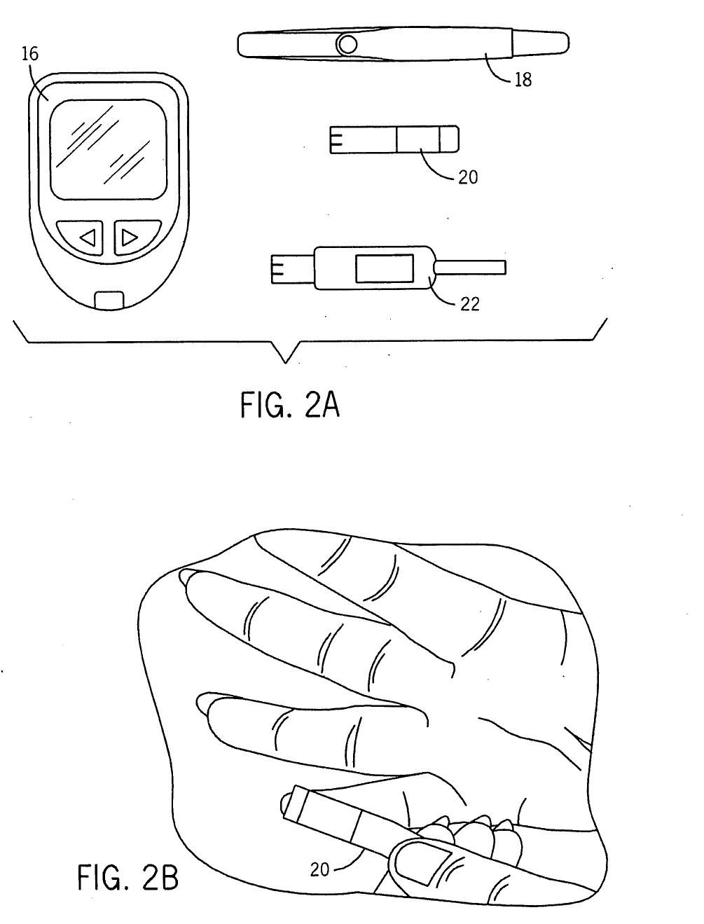

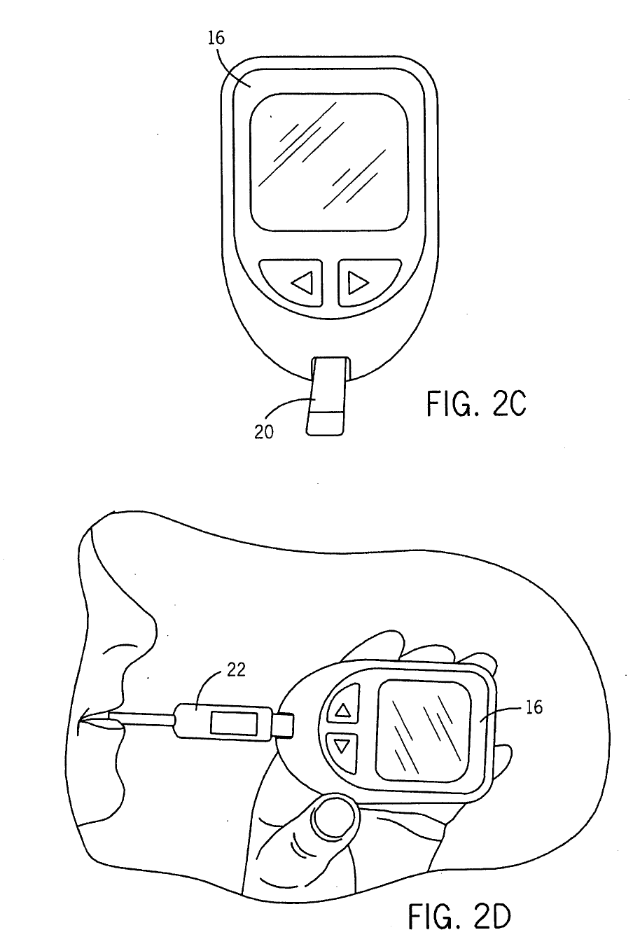

In some embodiments, the breath sensor 12 can be encased in a breath delivery system 22, as shown in FIGS. 2A-2D and 3. The breath delivery system 22 can include a mouthpiece (as shown in FIG. 2D) with a first end through which the individual can blow air so that air passes over the electrochemical biosensor. The breath delivery system shown and described in U.S. Provisional Patent Application No. 61/001,172 filed on Oct. 31, 2007, the entire contents of which is herein incorporated by reference, can be used in some embodiments of the biosensor system 10. Also, different types of sensors can be placed in the mouthpiece of the breath delivery system 22. In some embodiments, two different types of sensors can be placed in the mouthpiece and a switch can alter the air flow from one sensor chamber to another sensor chamber. For breath acetone analysis, a detection range can be about 0.1 ppm to about 100 ppm. For other analytes (e.g., to detect lung diseases or other breath biomarkers specific to disease states), a detection range can be less than about 0.1 ppm.

The breath delivery system 22 can include an opening in a second end of the mouthpiece for making an electrical connection to the portable electronic device 16. The breath delivery system 22 can be mechanically inserted into an opening in the housing of the portable electronic device 16. As shown in FIG. 3, the portable electronic device 16 can also include a suitable display 24.

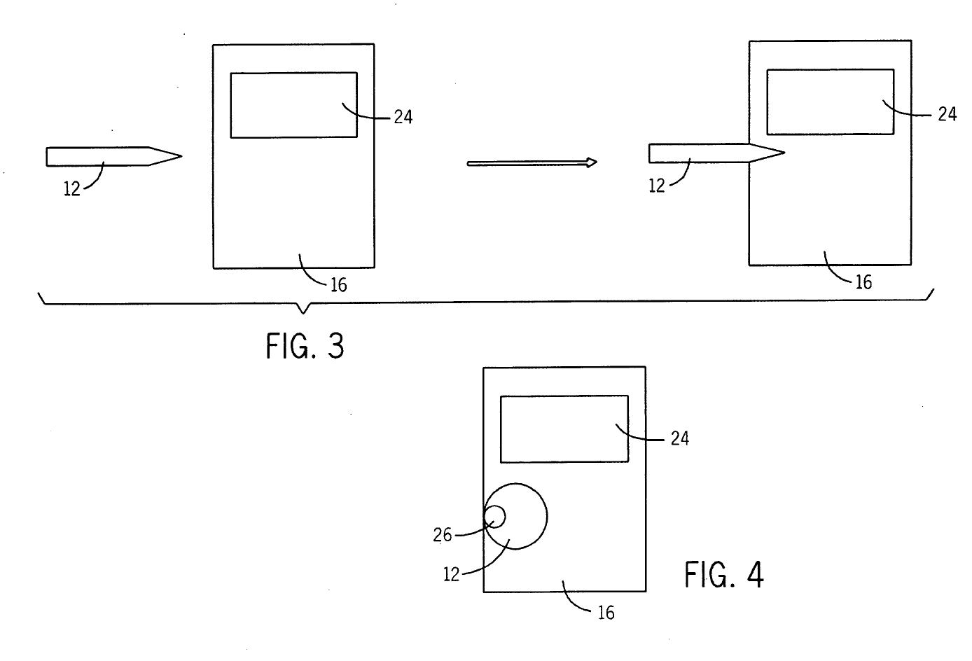

In other embodiments, as shown in FIG. 4, the breath sensor 12 can be embedded into the portable electronic device 16. The portable electronic device 16 can include the embedded breath sensor 12, the display 24, and a breath opening 26. Some embodiments of the invention allow for a single portable electronic device 16 to sense and measure multiple breath analytes or a combination of breath and blood or other analytes specific to a metabolic or disease state. Some embodiments of the invention provide the user a single portable electronic device 16 with multiple analyte detection (versus having multiple stand alone devices each measuring separate analyte or chemical). Some embodiments of the invention also allow the user to have a single portable electronic device 16 which is not restricted to analyte detection, but can be used with other functionality, such as cellular phone or personal digital assistant functions. The use of portable electronic devices continues to increase and more and more features are embedded into the portable electronic devices. Some embodiments of the invention add a new dimension or feature to the portable electronic device 16 for use in personal health and disease management. The breath sensing system 10 can improve the data collection process by allowing a single portable electronic device 16 to transmit or receive data to and from the physician and the individual via wireless or wired connections such as BlueTooth, IR, USB, etc. The breath sensing system 10 can also improve the time to administer medical therapies, assess compliance and provide data for insurance providers, individuals, and physicians.

In some embodiments, the portable electronic device 16 can include other embedded health or medical biosensing systems (e.g., a blood glucose meter), where data signal transmission and receipt may or may not occur. Some embodiments of the invention integrate a breath acetone biosensor with a glucose monitor for the tandem detection of breath acetone and blood glucose. As shown in FIGS. 2A-2D, a lancet 18 and a blood glucose strip 20 can be used along with the breath delivery system 22 to generate blood glucose data and breath acetone data that can be stored in the personal electronic device 16 and, in some embodiments, transmitted from the personal electronic device 16.

As many current portable glucose monitors measure only blood products, some embodiments of the invention provide a breath sensor 12 that is compatible for use with a blood glucose monitoring electrical connector in a portable electronic device 16 for data signal transmission and receipt (for example, the GlucoPhone cellular phone by HealthPia America with which blood glucose is monitored). Breath is a gaseous biomaterial. The breath delivery system 22 can be adaptable to a blood glucose-type electrode strip (such as disclosed in U.S. Pat. No. 6,609,068 issued Aug. 19, 2003, the entire contents of which is herein incorporated by reference) that is electrochemically designed to react with the desired analytes specific to a particular active chemistry. Some embodiments of the invention integrate a breath acetone biosensor (e.g., an enzymatic electrochemical biosensor, e-Nose type biosensors, thermally resistive biosensors, chemically resistive biosensors, etc.) with other health and medical sensors for measuring breath, blood, urine, or other physical or medical attributes, where the integrated device may or may not be capable of transmitting data.

The integration of the breath acetone biosensor into a blood glucose monitor provides the individual with dual functionality in a single portable electronic device 16 (versus multiple devices). In disease management, the majority of the type 2 diabetics are overweight and obese. Using the breath biosensor to monitor diet and fitness in a weight loss regimen combined with the common need for diabetics to test their blood sugar (blood glucose measurement) can provide the user with a single, well-rounded tool to control their health. The data can be sent to the individual's medical care professional for diagnostic, feedback and treatment, and medical therapies. The acetone breath sensor can be integrated with a blood glucose meter that uses blood, or that reads the eye, or that uses a laser through the finger. The latter two blood glucose measurements in tandem with breath acetone also provide a fuller and substantially noninvasive health management system (where data can be transmitted if desired).

Some embodiments of the invention include the transmission or receipt of data via the Internet or other methods specific to transmitting or receipt of wireless data from the portable electronic device 16 to a health or medical management system in order to provide the health/medical professional with information to diagnose, treat and care for the user (individual) of the biosensor system 10.

In another embodiment, the ability to sense breath acetone (electrochemically or by other means) in combination with other analytes permits the possibility of a dual function portable electronic device 16 that can transmit data. For example, sampling breath acetone and breath ammonia (NH₃) in a single device can allow differentiation between fat loss and muscle loss, respectively. This dual sensor set can also be integrated into a respirator mask in a critical care environment.

Some embodiments of the invention add utility to a portable electronic device 16 (such as a cellular phone or an iphone) and add functionality to the common glucose meter with a breath biosensor. Some embodiments of the invention also add multiple breath tests into a single portable device, including, for example, an electrochemical breath acetone test, and integrate portable and potentially data-transmittable breath acetone measurement with other breath and health measurement devices. The integration of multiple sensors provides a broader health picture in a single portable electronic device 16. The biosensor can detect breath analytes such as acetone, which is a biomarker for fat metabolism and disease states, such as diabetes and hyperthyroidism.

Some embodiments of the invention relate to the area of health, medical and disease management where detection, analysis and bioinformatics are used. The information from the biosensor can be transmitted to the individual's physician's database and treatments by the physician can be suggested remotely. It can also provide for remote medical care to monitor the efficacy of physician-controlled therapies. Interface software linked to devices similar to the MedApps system (www.medapps.net) are optional software/hardware and database-creation links between physicians and individuals. Also, the physician can create a database to track the individual or a specific individual population to develop more effective treatments.

Some embodiments of the invention can be used by cellular phone manufacturers, individuals who do not want to carry multiple portable electronic devices with them (this allows for a multifunctional single device), physicians who's patients are diabetic (specifically type 2 diabetic to monitor diet compliance), and physicians who perform weight loss surgeries to help monitor dietary compliance.

Multiple portable sensors including breath acetone sensing can be used in emergency rooms and/or respirator masks and/or long term care hospitals and/or during studies of metabolism and/or during studies of exercise and/or to monitor long term care patients, such as the elderly, HIV, cancer patients, and anorexics.

The following paragraphs describe several embodiments of a breath sensor 12 integrated with a multifunctional portable electronic device 16 that can, in some embodiments, analyze the data and electronically receive and transmit the outcome of the analysis of the breath components.

In one embodiment, as shown in FIG. 3, the breath sensor 12 is mechanically inserted through an opening forming an electrical connection between the breath sensor 12 and the portable electronic device 16. Breath from the individual activates the breath sensor 12 resulting in an electrical signal. The electrical signal is transmitted from the breath sensor 12 to the portable electronic device 16. The portable electronic device 16 can analyze the signal using a microcontroller or other data processing component and can then display the outcome on the display 24. The portable electronic device 16 can store the data and/or the outcome of the analysis in memory for future access by the individual or medical professional. The portable electronic device 16 can transmit the data and/or the outcome of the analysis to a software system used to track the individual's outcome. The portable electronic device 16 can transmit the data and/or the outcome of the analysis to a health or medical care professional for diagnostics, patient records, or medical therapy/treatment response. The breath sensor 12 can be removed from the portable electronic device 16 after being used.

In another embodiment, as shown in FIG. 4, the breath sensor 12 is mechanically and electrically embedded into the portable electronic device 16. An opening is designed into the portable electronic device 16 for the breath to be directed to the breath sensor 12. Breath from the individual can activate the sensor 12 resulting in an electrical signal. The electrical signal is transmitted from the breath sensor 12 to the portable electronic device 16. The portable electronic device 16 can store the data and/or the outcome of the analysis in memory for future access by the individual or medical professional. The portable electronic device 16 can transmit the data and/or the outcome of the analysis to a software system used to track the individual's outcome. The portable electronic device 16 can transmit the data and/or the outcome of the analysis to a health or medical care professional for diagnostics, patient records, or medical therapy/treatment response.

In another embodiment, the breath sensor 12 is inserted through an opening and forms an electrical connection between the breath sensor 12 and a multifunctional glucose and breath monitoring device 16. Breath from the individual can activate the breath sensor 12 resulting in an electrical signal. The electrical signal is transmitted from the breath sensor 12 to the monitoring device 16. The monitoring device 16 can store the data and/or the outcome of the analysis in memory for future access by the individual or medical professional. The monitoring device 16 can directly transmit the data and/or the outcome of the analysis to a software system used to track the individual's outcome. The monitoring device 16 can directly transmit the data and/or the outcome of the analysis to a health or medical care professional for diagnostics, patient records, or medical therapy/treatment response. The breath sensor 12 is removed from the monitor device 16 after being used.

In another embodiment, the breath sensor 12 is mechanically and electrically embedded into a dual-detection device such as a glucose monitoring and breath analyte monitoring device 16. An opening is designed into the monitoring device 16 for the breath to be directed to the breath sensor 12. Breath from the individual activates the breath sensor 12 resulting in an electrical signal. The electrical signal is transmitted from the breath sensor 12 to the monitoring device 16. The monitoring device 16 can store the data and/or the outcome of the analysis in memory for future access by the individual or medical professional. The monitoring device 16 can directly transmit the data and/or the outcome of the analysis to a software system used to track the individual's outcome. The monitoring device 16 can directly transmit the data and/or the outcome of the analysis to a health or medical care professional for diagnostics, patient records, or medical therapy/treatment response.

The following paragraphs describe examples of a breath acetone sensing device (e.g., palm-size electrochemical enzymatic sensing device) integrated with other breath sensing and/or other devices. Therefore, each of the following examples presents a single device that can provide a fuller picture of a individual's health status.

### EXAMPLE 1

A breath acetone sensor in tandem with blood glucose meter or pump, or with a non-invasive blood glucose measuring system, such as eye scan or infrared (IR) scan through the individual's finger. Integrating both sensors into a single device provides diabetics much useful information on their metabolic state in relation to their insulin control. Integrating the breath acetone sensor with non-invasive methods of blood glucose measurement can provide both measurements in a fully non-invasive device. The similarity of the design methods for electrochemical measurements in a breath acetone and a blood glucose meter make these devices particularly suitable to integrate. For example, the glucose electrode strip can be first used in the base unit, then replaced by inserting a mouthpiece with an electrochemical acetone biosensor using the same electrical and mechanical connection component as the glucose strip.

### EXAMPLE 2

A breath acetone sensor in tandem with resting metabolic rate measurement such as the BodyGem® by Microlife.

### EXAMPLE 3

A breath acetone sensor in tandem with respiratory quotient measurement or any other metabolic measurement.

### EXAMPLE 4

A breath acetone sensor in tandem with breath carbon dioxide (CO₂) measurement tool.

### EXAMPLE 5

A breath acetone sensor in tandem with any other breath component sensor [such as alcohol, ammonia (NH₃), urea, hydrogen, sulfides, nitric oxide (NO), isoprene, ethane, pentane, methanol, etc.] or any other breath condensate measurement. For example, breath acetone and breath NH₃ can provide a combined picture of fat metabolism with muscle breakdown for energy. This can be helpful for individuals monitoring their health after bariatric surgery, individuals with cachexia, or endurance athletes to avoid muscle wasting. NH₃ in the breath can also signal kidney failure, and this plus breath acetone can provide a more complete health picture. NH₃ sensor can be integrated into a current device using a replaceable NH₃ sensor, or by fixing the sensor and having the breath directed to a different sensor chamber when the NH₃ level is desired. The NH₃ sensor can be quartz microbalance, polymer, chemiresistive, tuning fork, etc. Nitrous oxide provides information about asthma, and hydrogen regarding digestive state. Urea is indicative of renal failure. Acetone in tandem with these items allows a person and/or physician to measure and monitor several body states in a single device.

### EXAMPLE 6

A breath acetone sensor in tandem with any blood measurement or measurement tool such as acetone, 3HB (3-hydroxybutyrate), acetoacetate, glucose, insulin, etc.

### EXAMPLE 7

A breath acetone sensor in tandem with urine acetoacetate or urine ketone measurement.

### EXAMPLE 8

A breath acetone sensor in tandem with heart rate monitor, such as those manufactured by Polar®, calorimeter or calorie counter, such as the Bodybugg™ by APEX, pedometer, accelerometer, speedometer, VO₂ max (which can be defined as the highest rate of oxygen consumption attainable during maximal or exhaustive exercise), or any other exercise-related measuring device.

### EXAMPLE 9

A breath acetone sensor in tandem with a scale.

### EXAMPLE 10

A breath acetone sensor in tandem with a fat/water ratio body measurement such as a body fat monitor, an impedance meter, or other similar device.

### EXAMPLE 11

A breath acetone sensor integrated into a respirator mask or similar assisted breathing device. This can provide information to the caregiver on the metabolic state of the individual. For example, persons with HIV or cachexia can be monitored for fat bum while they were being ventilated so that too high an acetone level can trigger adjustments to feeding.

### EXAMPLE 12

A breath acetone sensor in tandem with any of the above examples where the devices are connected to a computer, phone, or any other data transmittable device.

### EXAMPLE 13

A breath acetone sensor in tandem with an breath alcohol sensor.

### EXAMPLE 14

A breath NO sensor connected to a cellular phone can permit transmission of data regarding airway inflammation, such as occurs with asthma, to a healthcare provider.

### EXAMPLE 15

A transmittable device such as a cellular phone, walki-talki, iPod®, or other device with sensors to detect breath analytes or condensates or airborn contaminants. Data regarding air contamination, or the state of contaminated individuals, can be transmitted outside the contaminated area.

### EXAMPLE 16

A portable electronic transmittable device that detects breath acetone using an enzymatic electrochemical or other means plus isoprene (for cholesterol), pentane and ethane (for lipic peroxidaiton and oxidative stress), NO for asthma, COPD, and H₂ for digestive disorders.

### EXAMPLE 17

A breath acetone sensor in tandem with a wrist watch.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A biosensor system for use by an individual, the system comprising:
a breath delivery system including a breath sensor capable of detecting an analyte in the individual's breath; and
a portable electronic device capable of receiving breath analyte data from the breath sensor,
the portable electronic device capable of receiving additional personal health data,
the portable electronic device capable of at least one of storing, analyzing, and transmitting the breath analyte data and the additional personal health data.

2. The system of claim 1 wherein the analyte in the individual's breath is at least one of acetone, ammonia, carbon dioxide, alcohol, urea, hydrogen, sulfides, nitric oxide, isoprene, ethane, pentane, methanol, oxygen, acetaldehyde, hydrogen peroxide, glucose, formaldehyde, and volatile organic compounds.

3. The system of claim 1 wherein the additional personal health data includes at least one of metabolic rate data, respiratory data, blood data, urine data, exercise data, weight data, and impedance data.

4. The system of claim 3 wherein the blood data includes at least one of acetone, 3-hydroxybutyrate, acetoacetate, glucose, and insulin.

5. The system of claim 3 wherein the urine data includes at least one of urine acetoacetate and urine ketone.

6. The system of claim 3 wherein the exercise data is provided by at least one of a heart rate monitor, a pedometer, a speedometer, an accelerometer, a calorimeter, and an oxygen consumption versus intensity monitor.

7. The system of claim 3 wherein weight is measured by a scale and impedance is measured by a body fat monitor.

8. The system of claim 1 wherein the breath delivery system is included in a respirator mask.

9. A biosensor system for use by an individual, the system comprising:
a breath delivery system including a breath sensor capable of detecting an analyte in the individual's breath; and
a portable electronic device capable of receiving breath analyte data from the breath sensor,
the portable electronic device capable of receiving blood glucose data,
the portable electronic device capable of at least one of storing, analyzing, and transmitting the breath analyte data and the blood glucose data.

10. The system of claim 9 wherein the analyte in the individual's breath is acetone and the breath sensor is an enzymatic electrochemical biosensor.

11. The system of claim 9 wherein the portable electronic device analyzes the breath analyte data to determine one of a metabolic state and a disease state.

12. The system of claim 11 wherein the metabolic state is fat catabolism.

13. The system of claim 11 wherein the disease state is hyperthyroidism, ketoacidosis, and diabetes.

14. The system of claim 9 wherein the portable electronic device includes at least one of a laptop, a cellular phone, a personal digital assistant, a pocket personal computer, and a portable music player.

15. The system of claim 9 wherein the portable electronic device includes a blood glucose monitor that receives a blood glucose strip.

16. The system of claim 9 wherein the portable electronic device includes a noninvasive blood glucose monitor.

17. The system of claim 9 wherein the portable electronic device includes an opening to mechanically receive the breath delivery system.

18. The system of claim 17 wherein the opening includes an electrical connector to connect the breath delivery system to the portable electronic device.

19. The system of claim 9 wherein the breath delivery system is mechanically and electrically embedded into the portable electronic device.

20. The system of claim 19 wherein the breath delivery system includes a breath opening built into the portable electronic device and wherein the breath sensor is electrically embedded in the portable electronic device.

21. The system of claim 9 wherein the portable electronic device transmits the breath analyte data and the blood glucose data to at least one of a software system and a medical professional.
